(19) United States
(12) Patent Application Publication
(10) Pub. No.: US 2004/0002080 A1
(43) Pub. Date: Jan. 1, 2004

(54) Primers for use in detecting beta-lactamases

(75) Inventor: Nancy D. Hanson, Gretna, NE (US)

Correspondence Address: MUETING, RAASCH & GEBHARDT, P.A., P.O. BOX 581415, MINNEAPOLIS, MN, 55458, US

(73) Assignee: Creighton University, Omaha, NE

(21) Appl. No.: 10/318,970

(22) Filed: Dec. 13, 2002

Related U.S. Application Data
(60) Provisional application No. 60/340,466, filed on Dec. 14, 2001.

(51) Int. Cl. C12Q 1/68; C07H 21/04; C12P 19/34

(52) U.S. Cl. 435/6; 435/91.2; 536/24.3

(57) ABSTRACT

Oliognucleotide primers are provided that are specific for nucleic acid characteristic of certain beta-lactamases. The primers can be employed in methods to identify nucleic acid characteristic of family-specific beta-lactamase enzymes in samples, and particularly, in clinical isolates of Gram-negative bacteria.

This application claims the benefit of U.S. Provisional Application No. 60/340,466, filed Dec. 14, 2001, which is incorporated herein by reference in its entirety.

### BACKGROUND

A disturbing consequence of the use, and over-use, of beta-lactam antibiotics (e.g., penicillins and cephalosporins) has been the development and spread of new beta-lactamases. Beta-lactamases are enzymes that open the beta-lactam ring of penicillins, cephalosporins, and related compounds, to inactivate the antibiotic. The production of beta-lactamases is an important mechanism of resistance to beta-lactam antibiotics among Gram-negative bacteria.

Expanded-spectrum cephalosporins have been specifically designed to resist degradation by the older broad-spectrum beta-lactamases such as TEM-1, 2, and SHV-1. Microbial response to the expanded-spectrum cephalosporins has been the production of mutant forms of the older beta-lactamases called extended-spectrum beta-lactamases (ESBLs). Although ESBL-producing Enterobacteriaceae were first reported in Europe in 1983 and 1984, ESBLs have now been found in organisms of diverse genera recovered from patients in all continents except Antarctica. The occurrence of ESBL-producing organisms varies widely with some types more prevalent in Europe (TEM-3), others more prevalent in the United States (TEM-10, TEM-12 and TEM-26), while others appear worldwide (SHV-2 and SHV-5). These enzymes are capable of hydrolyzing the newer cephalosporins and aztreonam. Studies with biochemical and molecular techniques indicate that many ESBLs are derivatives of older TEM-1, TEM-2, or SHV-1 beta-lactamases, some differing from the parent enzyme by one or more acid substitutions.

Furthermore, carbapenem-hydrolyzing beta-lactamases are some of the more recently described beta-lactamases in the repertoire of penicillin-interactive proteins. These enzymes are responsible for conferring resistance to the carbapenems, the β-lactam class with the broadest spectrum of antibacterial activity, by preferentially hydrolyzing carbapenems (Rasmussen et al., Antimicrobial Agents and Chemotherapy, 41(2):223-232 (February 1997)). On a molecular level these enzymes can belong either to the class A group of beta-lactamases that have serine at the active site or to the class B enzymes that represent the only metallo-beta-lactamases identified (Ambler, Philos. Trans. R. Soc. London Biol., 289:321-331 (1980)).

In addition, resistance in Klebsiella pneumoniae and Escherichia coli to cephamycins and inhibitor compounds such as clavalante have also arisen via acquisition of plasmids containing chromosomally derived AmpC beta-lactamase genes, most commonly encoded by Enterobacter cloacae, Citrobacter freundii, Morganella morganiii, and Hafnia alvei.

It is of particular concern that genes encoding the beta-lactamases are often located on large plasmids that also contain genes for resistance to other antibiotic classes including aminoglycosides, tetracycline, sulfonamides, trimethoprim, and chloramphenicol. Furthermore there is an increasing tendency for pathogens to produce multiple beta-lactamases. These developments, which occur over a wide range of Gram-negative genera, represent a recent evolutionary development in which common Gram-negative pathogens are availing themselves of increasingly complex repertoires of antibiotic resistance mechanisms. Clinically, this increases the difficulty of identifying effective therapies for infected patients.

Thus, there is a need for techniques that can quickly and accurately identify the types of beta-lactamases that may be present in a clinical isolate or sample, for example. This could have significant implications in the choice of antibiotic necessary to treat a bacterial infection.

### SUMMARY OF THE INVENTION

The present invention is directed to the use of oligonucleotide primers specific to nucleic acids characteristic of (typically, genes encoding) certain beta-lactamase enzymes. More specifically, the present invention uses primers to identify family specific beta-lactamase nucleic acids (typically, genes) samples, particularly, in clinical isolates of Gram-negative bacteria. Specific primers of the invention include the primer sequences set forth in SEQ ID NOs: 1-39. As used herein, a nucleic acid characteristic of a beta-lactamase enzyme includes a gene or a portion thereof. A “gene” as used herein, is a segment or fragment of nucleic acid (e.g., a DNA molecule) involved in producing a peptide (e.g., a polypeptide and/or protein). A gene can include regions preceding (upstream) and following (downstream) a coding region (i.e., regulatory elements) as well as intervening sequences (introns, e.g., non-coding regions) between individual coding segments (exons). The term “coding region” is used broadly herein to mean a region capable of being transcribed to form an RNA, the transcribed RNA can be, but need not necessarily be, further processed to yield an mRNA.

Additionally, a method for identifying a beta-lactamase in a clinical sample is provided. Preferably, the clinical sample provided is characterized as a Gram-negative bacteria with resistance to beta-lactam antibiotics. The method includes, providing a pair of oligonucleotide primers, wherein one primer of the pair is complementary to at least a portion of the beta-lactamase nucleic acid in the sense strand and the other primer of each pair is complementary to a different portion of the beta-lactamase nucleic acid in the antisense strand; annealing the primers to the beta-lactamase nucleic acid; simultaneously extending the annealed primers from a 3′ terminus of each primer to synthesize an extension product complementary to the strands annealed to each primer wherein each extension product after separation from the beta-lactamase nucleic acid serves as a template for the synthesis of an extension product for the other primer of each pair; separating the amplified products; and analyzing the separated amplified products for a size characteristic of the type of beta-lactamase gene.

The method, described above, can employ oligonucleotide primers that are specific for nucleic acid of the AmpC family of beta-lactamases, the OXA-30 beta-lactamase, and the carbapenem-hydrolyzing family of beta-lactamases. Additional primers that can be used include those that are specific for nucleic acid of the AmpC beta-lactamases originating from Enterobacter cloacae, Citrobacter freundii, Serratia marcescens, Pseudomonas aeruginosa, Morganella morganii, and Hafnia alvei.

Still other oligonucleotide primers that are suitable for use in the method of the present invention include primers that are specific for nucleic acid of the plasmid-mediated AmpC beta-lactamases designated as FOX-1, DHA-1, DHA-2, ACC-1, MIR-1, or ACT-1; primers specific for nucleic acid of OXA-30 beta-lactamase; and primers specific for nucleic acid of class A carbapenem-hydrolyzing beta-lactamases.

The present invention is further directed to diagnostic kits for detecting beta-lactamases. The kit includes packaging, containing, separately packaged, at least one primer capable of hybridizing to beta-lactamase nucleic acid of interest, for instance, AmpC family of beta-lactamases, OXA-30 beta-lactamase, and carbapenem-hydrolyzing family of beta-lactamases; a positive and negative control, and a protocol for identification of the beta-lactamase nucleic acid of interest, wherein the primers are selected from the group consisting of SEQ ID NOs:1-39.

### BRIEF DESCRIPTION OF THE FIGURES

FIG. 1. Agarose gel showing a polymerase chain reaction (PCR) analysis of Citrobacter origin genes encoded by K. pneumoniae 249. Lanes 1 and 6, positive control amplification of the chromosomal ampC gene from C. freundii; lanes 2 and 4, amplification of template DNA from K. pneumoniae 249; lanes 3 and 5, positive control amplification of the pmampC gene of Citrobacter origin, LAT-1; and lane M, 100 base pair ladder.

### DETAILED DESCRIPTION OF PREFERRED EMBODIMENTS

The present invention is directed to the detection of nucleic acid that is characteristic of (e.g., at least a segment of a gene that codes for) family-specific beta-lactamase nucleic acid in samples (e.g., clinical isolates of Gram-negative bacteria). Specifically, the present invention is directed to the detection of beta-lactamase nucleic acid (preferably, a gene or at least a segment of a gene) using unique primers and the polymerase chain reaction. Using the primers and methods of the present invention, beta-lactamases belonging to Bush group 1 (AmpC), OXA-30 beta-lactamase, and carbapenem-hydrolyzing beta-lactamases for example, can be identified.

The primers and methods of the present invention are useful for a variety of purposes, including, for example, the identification of the primary beta-lactamase(s) responsible for resistance to third generation cephalosporins among Gram-negative bacteria such as Escherichia coli and Klebsiella pneumoniae (Thomson et al., Antimicrob. Agents Chemother., 36(9): 1877-1882 (1992)). Other sources of beta-lactamases include, for example, a wide range of Enterobacteriaceae, including Enterobacter spp., Citrobacter freundii, Morganella morganii, Providencia spp., and Serratia marcescens (Jones, Diag. Microbiol. Infect. Disease., 31(3):461-466 (1998)). Additional beta-lactamase gene sources include Pseudomonas aeruginosa (Patrice et al., Antimicrob. Agents Chemother., 37(5):962-969 (1993)); Proteus mirabilis (Bret et al., Antimicrob. Agents Chemother., 42(5): 1110-1114 (1998)); Yersinia enterocolitica (Barnaud et al., FEMS Microbiol. Letters, 148(1): 15-20 (1997)); and Klebsiella oxytoca ( Marchese et al., Antimicrob. Agents Chemother., 42(2):464-467 (1998)).

The methods of the present invention involve the use of the polymerase chain reaction sequence amplification method (PCR) using novel primers. U.S. Pat. No. 4,683,195 (Mullis et al.) describes a process for amplifying, detecting, and/or cloning nucleic acid. Preferably, this amplification method relates to the treatment of a sample containing nucleic acid (typically, DNA) of interest from bacteria, particularly Gram-negative bacteria, with a molar excess of an oligonucleotide primer pair, heating the sample containing the nucleic acid of interest to yield two single-stranded complementary nucleic acid strands, adding the primer pair to the sample containing the nucleic acid strands, allowing each primer to anneal to a particular strand under appropriate temperature conditions that permit hybridization, providing a molar excess of nucleotide triphosphates and polymerase to extend each primer to form a complementary extension product that can be employed in amplification of a desired nucleic acid, detecting the amplified nucleic acid, and analyzing the amplified nucleic acid for a size specific amplicon (as indicated below) characteristic of the specific beta-lactamase of interest. This process of heating, annealing, and synthesizing is repeated many times, and with each cycle the desired nucleic acid increases in abundance. Within a short period of time, it is possible to obtain a specific nucleic acid, e.g., a DNA molecule, that can be readily purified and identified.

The oligonucleotide primer pair includes one primer that is substantially complementary to at least a portion of a sense strand of the nucleic acid and one primer that is substantially complementary to at least a portion of an antisense strand of the nucleic acid. The process of forming extension products preferably involves simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair. The amplified products are preferably detected by size fractionization using gel electrophoresis. Variations of the method are described in U.S. Pat. No. 4,683,194 (Saiki et al.). The polymerase chain reaction sequence amplification method is also described by Saiki et al., Science, 230, 1350-1354 (1985) and Scharf et al., Science, 324, 163-166 (1986).

An “oligonucleotide,” as used herein, refers to a polymeric form of nucleotides of any length, either ribonucleotides or deoxyribonucleotides. The term oligonucleotide refers particularly to the primary structure, and thus includes double and single-stranded DNA molecules and double and single-stranded RNA molecules.

A “primer,” as used herein, is an oligonucleotide that is complementary to at least a portion of nucleic acid of interest and, after hybridization to the nucleic acid, may serve as a starting-point for the polymerase chain reaction. The terms “primer” or “oligonucleotide primer,” as used herein, further refer to a primer, having a nucleotide sequence that possesses a high degree of nucleic acid sequence similarity to at least a portion of the nucleic acid of interest. “High degree” of sequence similarity refers to a primer that typically has at least about 80% nucleic acid sequence similarity, and preferably at least about 90% nucleic acid sequence similarity. Sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

The terms “complement” and “complementary” as used herein, refer to a nucleic acid that is capable of hybridizing to a specified nucleic acid molecule under stringent hybridization conditions. Stringent hybridization conditions include, for example, temperatures from about 50 degrees Celsius (° C.) to about 65° C., and magnesium chloride (MgCl₂) concentrations from about 1.5 millimolar (mM) to about 2.0 mM. Thus, a specified DNA molecule is typically “complementary” to a nucleic acid if hybridization occurs between the specified DNA molecule and the nucleic acid. If the specified DNA molecule hybridizes to the full-length of the nucleic acid molecule, then the specified DNA molecule is typically a “full-length complement.” “Complementary” further refers to the capacity of purine and pyrimidine nucleotides to associate through hydrogen bonding in double stranded nucleic acid molecules. The following base pairs are complementary: guanine and cytosine; adenine and thymine; and adenine and uracil.

As used herein, the terms “amplified molecule,” “amplified fragment,” and “amplicon” refer to a nucleic acid molecule (typically, DNA) that is a copy of at least a portion of the nucleic acid and its complementary sequence. The copies correspond in nucleotide sequence to the original molecule and its complementary sequence. The amplicon can be detected and analyzed by a wide variety of methods. These include, for example, gel electrophoresis, single strand conformational polymorphism (SSCP), restriction fragment length polymorphism (RFLP), capillary zone electrophoresis (CZE), and the like. Preferably, the amplicon can be detected, and hence, the type of beta-lactamase identified, using gel electrophoresis and appropriately sized markers, according to techniques known to one of skill in the art.

The primers are oligonucleotides, either synthetic or naturally occurring, capable of acting as a point of initiating synthesis of a product complementary to the region of the DNA molecule containing the beta-lactamase of interest. The primer includes nucleotides capable of hybridizing under stringent conditions to at least a portion of at least one strand of a nucleic acid molecule of a given beta-lactamase. Preferably, the primers of the present invention typically have at least about 12 nucleotides and more preferably at least about 15 nucleotides. Preferably, the primers have no more than about 35 nucleotides, and more preferably, no more than about 25 nucleotides. The primers are chosen such that they preferably produce a primed product of about 150-1300 base pairs.

Optionally, a primer used in accordance with the present invention includes a label constituent. The label constituent can be selected from the group of an isotopic label, a fluorescent label, a polypeptide label, and a dye release compound. The label constituent is typically incorporated in the primer by including a nucleotide having the label attached thereto. Isotopic labels preferably include those compounds that are beta, gamma, or alpha emitters, more preferably isotopic labels are selected from the group of ³²P, ³⁵S, and ¹²⁵I. Fluorescent labels are typically dye compounds that emit visible radiation in passing from a higher to a lower electronic state, typically in which the time interval between adsorption and emission of energy is relatively short, generally on the order of about 10⁻⁸ to about 10⁻³ second. Suitable fluorescent compounds that can be utilized include fluorescien and rhodamine, for example. Suitable polypeptide labels that can be utilized in accordance with the present invention include antigens (e.g., biotin, digoxigenin, and the like) and enzymes (e.g., horse radish peroxidase). A dye release compound typically includes chemiluminescent systems defined as the emission of absorbed energy (typically as light) due to a chemical reaction of the components of the system, including oxyluminescence in which light is produced by chemical reactions involving oxygen.

Preferred examples of these primers, that are specific for certain beta-lactamases, including flanking sequences, are as follows, wherein “F” in the designations of the primers refers to a 5′ upstream primer and “R” refers to a 3′ downstream primer. For those beta-lactamases that have more than one upstream primer and more than one downstream primer listed below as preferred primers, various combinations can be used. Typically, hybridization conditions utilizing primers of the invention include, for example, a hybridization temperature of about 40° C. to about 60° C., and a MgCl₂ concentration of about 1.5 mM to about 2.0 mM. Although lower or higher temperatures and higher concentrations of MgCl₂ can be employed, this may result in decreased primer specificity.

The following primers are specific for nucleic acid characteristic of the OXA-30 beta-lactamase enzyme (preferably flanking sequences).

| | |
|---|---|
| Primer Name: | |
| OXA305F | |
| Primer Sequence: | |
| 5′-GGAGCAGCAACGATGTTACG-3′ | (SEQ ID NO:1) |
| Primer Name: | |
| OXA303R | |
| Primer Sequence: | |
| 5′-CGACTTGATTGAAGGGTTGG-3′ | (SEQ ID NO:2) |

Employing a primer pair containing the primer sequences of SEQ ID NO:1 and SEQ ID NO:2 to a sample known to contain a OXA-30 beta-lactamase, a size-specific amplicon of 989 base pairs will typically be obtained.

The following primers are specific for nucleic acid characteristic of the AmpC beta-lactamase enzyme originating from Pseudomonas

| | |
|---|---|
| Primer Name: | |
| PaampCTF | |
| Primer Sequence: | |
| 5′-GCATTACTTCAGCTATGGGC-3′ | (SEQ ID NO:3) |
| Primer Name: | |
| PaampCTR | |
| Primer Sequence: | |
| 5′-GGCATTGGGATAGTTGCGGTTG-3′ | (SEQ ID NO:4) |

Employing a primer pair containing the primer sequences of SEQ ID NO:3 and SEQ ID NO:4 to a sample known to contain an AmpC beta-lactamase originating from Pseudomonas aeruginosa, a size-specific amplicon of 931 base pairs will typically be found.

The following primers are specific for nucleic acid characteristic of the plasmid-mediated AmpC beta-lactamase enzyme designated as FOX-1, FOX-2, or FOX-5 (preferably flanking sequences).

| | |
|---|---|
| Primer Name: | |
| FOXUP1F | |
| Primer Sequence: | |
| 5′-CACCACGAGAATAACC-3′ | (SEQ ID NO:5) |
| Primer Name: | |
| FOXD1R | |
| Primer Sequence: | |
| 5′-GCCTTGAACTCGACCG-3′ | (SEQ ID NO:6) |

Employing a primer pair containing the primer sequences of SEQ ID NO:5 and SEQ ID NO:6 to a sample known to contain a plasmid-mediated AmpC beta-lactamase, such as FOX-1, FOX-2, or FOX-5, a size-specific amplicon of 1,184 base pairs will typically be obtained.

The following primers are specific for nucleic acid characteristic of the AmpC beta-lactamase enzymes (both chromosomal and plasmid-mediated) originating from Citrobacter freundii.

| | |
|---|---|
| Primer Name: | |
| CMY25F1 | |
| Primer Sequence: | |
| 5′-CAATGTGTGAGAAGCAGTC-3′ | (SEQ ID NO:7) |
| Primer Name: | |
| CMYDR1 | |
| Primer Sequence: | |
| 5′-CGCATGGGATTTTCCTTGCTG-3′ | (SEQ ID NO:8) |
| Primer Name: | |
| CMY71F | |
| Primer Sequence: | |
| 5′-CGTTATGCTGCGCTCTGCTG-3′ | (SEQ ID NO:9) |
| Primer Name: | |
| CMY71R | |
| Primer Sequence: | |
| 5′-CTGCGGAACCGTAATCCAGG-3′ | (SEQ ID NO:10) |
| Primer Name: | |
| LATF2 | |
| Primer Sequence: | |
| 5′-CGTTATGCTGCGCTCTGC-3′ | (SEQ ID NO:11) |
| Primer Name: | |
| LATR2 | |
| Primer Sequence: | |
| 5′-GGCGATATCGGCTTTACC-3′ | (SEQ ID NO:12) |

Employing a primer pair containing the primer sequences of SEQ ID NO:7 and SEQ ID NO:8 to a sample known to contain the AmpC beta-lactamase genes CMY-2, CMY-5, and CMY-7 of Citrobacter freundii origin, a size-specific amplicon of 1,432 base pairs will typically be found. Employing a primer pair containing the primer sequences of SEQ ID NO:9 and SEQ ID NO:10 to a sample known to contain the AmpC beta-lactamases CMY-2, CMY-3, CMY-4, CMY-5, CMY-6, CMY-7, BIL-1, LAT-3, and LAT-4, a size-specific amplicon of 626 base pairs will typically be found. Employing a primer pair containing the primer sequences of SEQ ID NO:11 and SEQ ID NO:12 to a sample known to contain the AmpC beta-lactamases CMY-2, CMY-3, CMY-4, CMY-5, CMY-6, CMY-7, BIL-1, LAT-2, LAT-3, and LAT-4, a size-specific amplicon of 194 base pairs will typically be found.

The following primers are specific for nucleic acid characteristic of the AmpC beta-lactamase enzyme (both chromosomal and plasmid-mediated) originating from Morganella morganii.

| | |
|---|---|
| Primer Name: | |
| DHA1F | |
| Primer Sequence: | |
| 5′-CCGTTACTCACACACGGAAGG-3′ | (SEQ ID NO:13) |
| Primer Name: | |
| DHA1R | |
| Primer Sequence: | |
| 5′-CGTATCCGCAGGGGCCTGTTC-3′ | (SEQ ID NO:14) |
| Primer Name: | |
| MORGI1F | |
| Primer Sequence: | |
| 5′-GCGTCTGTATGCAAACAGCAG-3′ | (SEQ ID NO:15) |
| Primer Name: | |
| MORGI1R | |
| Primer Sequence: | |
| 5′-CAATGCGACCTCGTTGGTCACG-3′ | (SEQ ID NO:16) |

Employing a primer pair containing the primer sequences of SEQ ID NO:13 and SEQ ID NO:14 to a sample known to contain AmpC beta-lactamases DHA-1 and DHA-2, a size-specific amplicon of 1,199 and 1,200 base pairs, respectively, will typically be found. Employing a primer pair containing the primer sequences of SEQ ID NO:15 and SEQ ID NO:16 to a sample known to contain AmpC beta-lactamases DHA-1 and DHA-2, a size-specific amplicon of 439 base pairs will typically be found.

The following primers are specific for nucleic acid characteristic of the AmpC beta-lactamase enzyme (both chromosomal and plasmid-mediated) originating from Hafnia alvei.

| | |
|---|---|
| Primer Name: | |
| HFCFF1 | |
| Primer Sequence: | |
| 5′-CCGATTAAAAGGTCAC-3′ | (SEQ ID NO:17) |
| Primer Name: | |
| HFCFR1 | |
| Primer Sequence: | |
| 5′-GTGACTCAACATATCG-3′ | (SEQ ID NO: 18) |
| Primer Name: | |
| HFCIF1 | |
| Primer Sequence: | |
| 5′-CGTTAGCGTACTCAATGTGG-3′ | (SEQ ID NO:19) |
| Primer Name: | |
| HFCIR1 | |
| Primer Sequence: | |
| 5′-GATCCTGAGTAATCTCACCC-3′ | (SEQ ID NO:20) |

Employing a primer pair containing the primer sequences of SEQ ID NO:17 and SEQ ID NO:18 to a sample known to contain an AmpC beta-lactamase originating from Hafnia alvei, a size-specific amplicon of 1,244 base pairs will typically be found. Employing a primer pair containing the primer sequences of SEQ ID NO:19 and SEQ ID NO:20 to a sample known to contain an AmpC beta-lactamase originating from Hafnia alvei, a size-specific amplicon of 500 base pairs will typically be found.

The following primers are specific for nucleic acid characteristic of the AmpC beta-lactamase enzyme originating from Serratia marcescens.

| | |
|---|---|
| Primer Name: | |
| SDMCF | |
| Primer Sequence: | |
| 5′-CCTGCAACCTAAGAGAGCTTCT-3′ | (SEQ ID NO:21) |
| Primer Name: | |
| SDMCR | |
| Primer Sequence: | |
| 5′-CGCGGTGGATGATGTGGTAA-3′ | (SEQ ID NO:22) |

Employing a primer pair containing the primer sequences of SEQ ID NO:21 and SEQ ID NO:22 to a sample known to contain an AmpC beta-lactamase originating from Serratia marcescens, a size-specific amplicon of 1,140 base pairs will typically be found.

The following primers are specific for nucleic acid characteristic of the AmpC beta-lactamase enzyme (both chromosomal and plasmid-mediated) originating from Enterobacter cloacae.

| | |
|---|---|
| Primer Name: | |
| ACTPROBEF | |
| Primer Sequence: | |
| 5′-CCGGATGAGGTCAAGGATAACGC-3′ | (SEQ ID NO:23) |
| Primer Name: | |
| ACTPROBER | |
| Primer Name: | |
| 5′-CCCCAGGCGTAATGCGCCTCTTCC-3′ | (SEQ ID NO:24) |

Employing a primer pair containing the primer sequences of SEQ ID NO:23 and SEQ ID NO:24 to a sample known to contain an AmpC beta-lactamase originating from Enterobacter cloacae, a size-specific amplicon of 219 base pairs will typically be found.

The following primers are specific for nucleic acid characteristic of the class A carbapenem-hydrolyzing beta-lactamase enzyme.

| | |
|---|---|
| Primer Name: | |
| KPC-1F | |
| Primer Sequence: | |
| 5′-GCTACACCTAGCTCCACCTTC-3′ | (SEQ ID NO:25) |
| Primer Name: | |
| KPC-1R | |
| Primer Sequence: | |
| 5′-GACAGTGGTTGGTAATCCATGC-3′ | (SEQ ID NO:26) |
| Primer Name: | |
| KPCF-2 | |
| Primer Sequence: | |
| 5′-GTATCGCCGTCTAGTTCTGC-3′ | (SEQ ID NO:27) |
| Primer Name: | |
| KPCR-2 | |
| Primer Sequence: | |
| 5′-GGTCGTGTTTCCCTTTAGCC-3′ | (SEQ ID NO:28) |
| Primer Name: | |
| NMCAF1 | |
| Primer Sequence: | |
| 5′-GGTAATCTGGCACGCATGGT-3′ | (SEQ ID NO:29) |
| Primer Name: | |
| NMCAR1 | |
| Primer Sequence: | |
| 5′-CACACTGAGCATATGCTGAC-3′ | (SEQ ID NO:30) |
| Primer Name: | |
| IMIAIF1 | |
| Primer Sequence: | |
| 5′-GGCCAATACAAAGGGCATCG-3′ | (SEQ ID NO:31) |
| Primer Name: | |
| IMIAIR1 | |
| Primer Sequence: | |
| 5′-CTACCCAATCGCTTGGTACG-3′ | (SEQ ID NO:32) |

Employing a primer pair containing the primer sequences of SEQ ID NO:25 and SEQ ID NO:26 to a sample known to contain a KPC-1 class A carbapenem-hydrolyzing beta-lactamase, or derivatives thereof, a size-specific amplicon of 990 base pairs will typically be found. Employing a primer pair containing the primer sequences of SEQ ID NO:27 and SEQ ID NO:28 to a sample known to contain a KPC-1 and/or a KPC-2 class A carbapenem-hydrolyzing beta-lactamase, or derivatives thereof, a size-specific amplicon of 637 base pairs will typically be found. Employing a primer pair containing the primer sequences of SEQ ID NO:29 and SEQ ID NO:30 to a sample known to contain a NMC-A class A carbapenem-hydrolyzing beta-lactamase, or derivatives thereof, a size-specific amplicon of 1,151 base pairs will typically be found. Employing a primer pair containing the primer sequences of SEQ ID NO:31 and SEQ ID NO:32 to a sample known to contain an IMI-1 class A carbapenem-hydrolyzing beta-lactamase, or derivatives thereof, a size-specific amplicon of 620 base pairs will typically be found.

Various other primers, or variations of the primers described above, can also be prepared and used according to methods of the present invention. For example, alternative primers can be designed based on targeted beta-lactamases known or suspected to contain regions possessing high G/C content (i.e., the percentage of guanine and cytosine residues). As used herein, a “high G/C content” in a target nucleic acid, typically includes regions having a percentage of guanine and cytosine residues of about 60% to about 90%. Thus, changes in a prepared primer will alter, for example, the hybridization or annealing temperatures of the primer, the size of the primer employed, and the sequence of the specific resistance gene or nucleic acid to be identified. Therefore, manipulation of the G/C content, e.g., increasing or decreasing, of a primer or primer pair may be beneficial in increasing detection sensitivity in the method.

Additionally, depending on the suspected nucleic acid in the sample, a primer of the invention can be prepared that varies in size. Typically, primers of the invention are about 12 nucleotides to about 35 nucleotides in length, preferably the primers are about 15 nucleotides to about 25 nucleotides in length. Oligonucleotides of the invention can readily be synthesized by techniques known in the art (see, for example, Crea et al., Proc. Natl. Acad. Sci. (U.S.A.), 75:5765 (1978)).

Once the primers are designed, their specificity can be tested using the following method. Depending on the target nucleic acid of clinical interest, nucleic acid template is prepared from a bacterial control strain known to express or contain the resistance gene. This control strain, as used herein, refers to a “positive control” nucleic acid (typically, DNA). Additionally, a “negative control” nucleic acid (typically, DNA) can be isolated from one or more bacterial strains known to express a resistance gene other than the target gene of interest. Using the polymerase chain reaction, the designed primers are employed in a detection method, as described above, and used in the positive and negative control samples and in at least one test sample suspected of containing the resistance gene of interest. The positive and negative controls provide an effective and qualitative (or grossly quantitative) means by which to establish the presence or the absence of the gene of interest of test clinical samples. It should be recognized that with a small percentage of primer pairs, possible cross-reactivity with other Beta-lactamase genes might be observed. However, the size and/or intensity of any cross-reactive amplified product will be considerably different and can therefore be readily evaluated and dismissed as a negative result.

The invention also relates to kits for identifying family specific beta-lactamase enzymes by PCR analysis. Kits of the invention typically include one or more primer pairs specific for a beta-lactamase of interest, one or more positive controls, one or more negative controls, and protocol for identification of the beta-lactamase of interest using polymerase chain reaction. A negative control includes a nucleic acid (typically, DNA) molecule encoding a resistant beta-lactamase other than the beta-lactamase of interest. The negative control nucleic acid may be a naked nucleic acid (typically, DNA) molecule or be inserted into a bacterial cell. Preferably, the negative control nucleic acid is double stranded; however, a single stranded nucleic acid may be employed. A positive control includes a nucleic acid (typically, DNA) that encodes a beta-lactamase from the family of beta-lactamases of interest. The positive control nucleic acid may be a naked nucleic acid molecule or be inserted into a bacterial cell, for example. Preferably, the positive control nucleic acid is double stranded; however, a single stranded nucleic acid may be employed. Typically, the nucleic acid is obtained from a bacterial lysate.

Accordingly, the present invention provides a kit for characterizing and identifying a family specific beta-lactamase that would have general applicability. Preferably, the kit includes a polymerase (typically, DNA polymerase) enzyme, such as Taq polymerase, and the like. A kit of the invention also preferably includes at least one primer pair that is specific for a beta-lactamase gene or flanking regions of the gene. A buffer system compatible with the polymerase enzyme is also included and is well known in the art. Optionally, at least one primer pair may contain a labeled constituent, a fluorescent label, a polypeptide label, and a dye release compound. The kit may further contain at least one internal sample control, in addition to one or more further means required for PCR analysis, such as a reaction vessel. If required, a nucleic acid from the bacterial sample can be isolated and then subjected to PCR analysis using the provided primer set of the invention.

In another embodiment, family specific beta-lactamase enzymes in clinical samples, particularly clinical samples containing Gram-negative bacteria, can be detected by the primers described herein in a “microchip” detection method. In a microchip detection method, nucleic acid, e.g., genes, of multiple beta-lactamases in clinical samples can be detected with a minimal requirement for human intervention. Techniques borrowed from the microelectronics industry are particularly suitable to these ends. For example, micromachining and photolithographic procedures are capable of producing multiple parallel microscopic scale components on a single chip substrate. Materials can be mass produced and reproducibility is exceptional. The microscopic sizes minimize material requirements. Thus, human manipulations can be minimized by designing a microchip type surface capable of immobilizing a plurality of primers of the invention on the microchip surface.

Microchip detection methods generally include the formation of high-density arrays of, for example, oligonucleotides on a surface, typically glass, that can then be used for various applications, such as large scale hybridization (Roth et al., Annu. Rev. Biomed. Eng., 1:265-297 (1999)). Using this method, applications using two types of nucleic acids as targets, synthesizing or printing directly on a surface, or covalent or noncovalent attachment of single stranded cDNA, are known.

According to one of the methods of microchip detection, arrays of short oligonucleotides may be synthesized directly on a surface, such as glass, using methods generally known in solid-phase chemistry synthesis. This is generally accomplished by either masking most of the array, activating the unmasked portion, and adding a phosphoramidite to produce a coupling to the 5′-hydroxy groups of the activated segments of the array or by printing the arrays directly on the surface using ink jet printing techniques.

Alternatively, for production of longer sequences, such as cDNA arrays, a spotting method for attaching molecules to a surface may be used. In this method, for instance, sequences are created from clones, purified, and optionally amplified. The sequences are then attached noncovalently to a glass surface by coating the surface with polylysine or by chemically treating it with an aminosilane to make it cationic. Attachment of the sequences under this method are generally nonspecific and may involve multiple attachment sites along the molecule. An additional method known for attachment of sequences to a surface is to covalently attach amino-modified cDNA, produced by asymmetric PCR, to silylated glass using sodium borohydhydride (Roth et al., Annu. Rev. Biomed. Eng., 1:265-297 (1999)).

Thus, an object of the present invention is to provide a parallel screening method wherein multiple serial reactions are automatically performed individually within one reaction well for each of the plurality of nucleic acid strands to be detected in the plural parallel sample wells. These serial reactions are performed in a simultaneous run within each of the multiple parallel lanes of the device. “Parallel” as used herein means wells identical in function. “Simultaneous” means within one preprogrammed run. The multiple reactions automatically performed within the same apparatus minimize sample manipulation and labor.

Thus, the present invention provides multiple reaction wells, the reaction wells being reaction chambers, on a microchip, each reaction well containing an individualized array to be used for detecting a beta-lactamase gene uniquely specified by the substrates provided, the reaction conditions and the sequence of reactions in that well. The chip can thus be used as a method for identifying beta-lactamase genes in clinical samples.

Objects and advantages of this invention are further illustrated by the following examples, but the particular materials and amounts thereof recited in these examples, as well as other conditions and details, should not be construed to unduly limit this invention.

### EXAMPLE 1

### Molecular Identification of the CMY-2 Plasmid-mediated AmpC Beta-Lactamase from a Klebsiella pneumoniae Isolated from a Pediatric Patient

K. pneumoniae strain 249 was isolated from the urine of a 6-year-old girl with meningomyelocele and an associated neurogenic bladder. She was not a hospital inpatient at the time, nor had she been recently hospitalized. Renal ultrasound performed at the time showed no hydronephrosis or enlargement of the kidneys. She had a history of urinary tract infections and was not undergoing routine urinary catheterizations. Nitrofurantoin had been prescribed for an E. coli urinary tract infection 4 weeks prior, and she was being treated with nitrofurantoin at the time of presentation. She presented to a spinal defects clinic with a history of dysuria, but no fever. A catheterized urine specimen was obtained and the urine culture grew N greater than (>) 100,000 colony forming units per milliliter (cfu/mL) of K. pneumoniae. She was empirically treated with oral trimethoprim sulfamethoxazole prior to culture results and was later switched to ciprofloxacin. She was asymptomatic at a follow up visit 1 month later, however K. pneumoniae was still present when her urine was cultured.

Susceptibility tests were performed on K. pneumoniae 249 by an overnight microdilution method with investigational dehydrated panels provided by Dade Behring MicroScan (Sacramento, Calif.) according to the manufacturer's recommendations and interpreted according to National Committee for Clinical Laboratory Standards (NCCLS) criteria and by NCCLS disc diffusion (National Committee for Clinical Laboratory Standards (NCCLS), “Methods for Dilution Antimicrobial Susceptibility Tests for Bacteria that Grow Aerobically: Approved Standard M7-A4,” Villanova, Pa. (1997); and Hanson et al., J. Antimicrob. Chemother., 44(3):377-380 (September 1999)). The test was performed using an innoculum of approximately 5×10⁵ colony forming units per square milliliter (cfu/mL²). Isoelectric focusing (IEF), cefotaxime hydrolysis and inhibitor determinations in polyacrylamide gels were performed using sonic extracts of K. pneumoniae 249 (Thomson et al., Antimicrob. Agents Chemother., 35:1001-1003 (1991)).

Initial susceptibility studies on K. pneumoniae strain 249 indicated multiple β-lactam resistance. The Minimum Inhibitory Concentrations (MICs) were as follows: >64 micrograms per milliliter (μg/mL) of ticarcillin, and piperacillin, 64/2 μg/mL ticarcillin/clavulanate, >32 μg/mL cefoxitin, >16 μg/mL of ampicillin and cephalothin, 16 μg/mL ceftriaxone and ceftazidime, 16/2 μg/mL ceftriaxone/clavulanate and ceftazidime/clavulanate, >4 μg/mL of cefpodoxime, less than or equal to (≦) 4 μg/mL cefepime, and ≦0.5 μg/mL of meropenem. The cefoxitin, ceftriaxone/clavulanate and ceftazidime/clavulanate MICs indicated two possible mechanisms of resistance, the expression of a plasmid-mediated ampC gene and/or a porin mutation. The presence of an AmpC-type beta-lactamase was verified by a combination of isoelectric focusing (IEF), cefotaxime hydrolysis, and beta-lactamase inhibitor determinations in polyacrylamide gels using sonic extracts of K. pneumoniae 249 (Thomson et al., Antimicrob. Agents and Chemother., 35(5): 1001-1003 (May 1991); and Hanson et al., J. Antimicrob. Chemother., 44(3):377-380 (September 1999)). Three beta-lactamases were identified with isoelectric points of 5.4, 7.6, and 9.1. The band that focused at isoelectric point 9.1 hydrolyzed cefotaxime and was inhibited by cloxacillin (1000 mM), but not clavulanate (1000 mM). These findings indicated it was an AmpC enzyme.

Determination of the type of AmpC beta-lactamase present in this Klebsiella isolate required PCR and sequence analysis (Hanson et al., J. Antimicrob. Chemother., 44(3):377-380 (September 1999); Pitout et al., Antimicrob. Agents and Chemother., 42(6):1350-1354 (June 1998)). Template DNA preparation and PCR amplifications were carried out as follows:

The organisms were inoculated into 5 milliliters (mL) of Luria-Bertani broth (Difco, Detroit, Mich.) and incubated for 20 hours at 37° C. with shaking. Cells from 1.5 mL of an overnight culture were harvested by centrifugation at 17,310×g in a Hermle centrifuge (Hermle Company, Gosheim, Germany) for 5 minutes. After the supernatant was decanted, the pellet was resuspended in 500 microliters (μL) of distilled water. The cells were lysed by heating at 95° C. for 10 minutes, and cellular debris was removed by centrifugation at 17,310×g for 5 minutes. The supernatant was used as a source of template for amplification.

PCR amplifications were carried out on a DNA Thermal Cycler 480 instrument (Perkin-Elmer, Cetus, Norwalk, Conn.) with the Gene Amp DNA amplification kit containing AmpliTaq polymerase (Perkin-Elmer, Roche Molecular Systems, Inc., Branchburg, N.J.). The composition of the reaction mixture was as follows: 10 mM Tris-HCl (pH 8.3), 50 mM KCl, 1.5 mM MgCl₂, each of the four deoxynucleoside triphosphates at a concentration of 0.2 mM and 1.2 units (U) of AmpliTaq in a total volume of 49 μl. A total of 1 μl of sample lysate was added to the reaction mixture, the mixture was centrifuged briefly, and 50 μl of mineral oil was layered onto the surface. The PCR program consisted of an initial denaturation step at 96° C. for 15 seconds, followed by 24 cycles of DNA denaturation at 96° C. for 15 seconds, primer annealing at 50° C. for 15 seconds, and primer extension at 72° C. for 2 minutes.

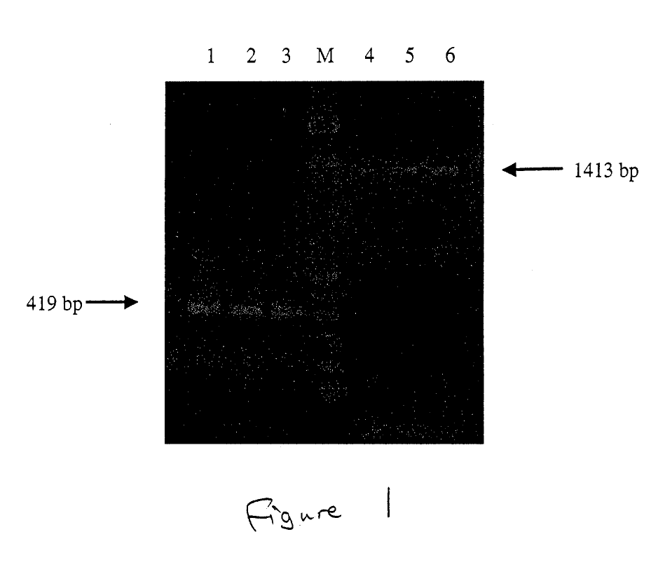

PCR analysis was used to confirm the presence and family-type of the plasmid-mediated ampC gene. PCR using an internal primer set specific for C. freundii (primer set 1) (forward primer 1: 5′-CTGGCAACCACAATGGACTCCG-3′ (SEQ ID NO:36) representing nucleotides (nts) 513-534 and reverse primer 1: 5′-GCCAGTTCAGCATCTCCCAGCC-3′ (SEQ ID NO:37) representing nts 931-910) amplified a 419 base pair (bp) fragment (FIG. 1, lane 2) (Lindberg et al., Eur. J. Biochem., 156:441-445 (1986)). In order to identify the entire sequence of the plasmid-mediated ampC gene, it was amplified using a primer set designed to flank the structural gene of plasmid-mediated ampC genes of C. freundii origin (primer set 2). Primer set 2 (forward 2: 5′-CAATGTGTGAGAAGCAGTC-3′ (SEQ ID NO:7) 3′ corresponding to nts 1736-1754 and reverse 2: 5′-CGCATGGGATTTTCCTTGCTG-3′ (SEQ ID NO:8) corresponding to nts 3167-3147 amplified a 1413 bp fragment (FIG. 1, lane 4) (Bauernfeind et al., Antimicrob Agents Chemother. 1996;40:221-224).

The fragment amplified by primer set 2 was directly sequenced in both directions at least two separate times. Sequencing was performed by PCR cycle sequencing with dye-terminator chemistry using a DNA stretch sequencer from Applied Biosystems (Foster City, Calif., USA). Nucleotide and protein sequence alignments were performed on-line using BLAST (National Center for Biotechnology Information, http:H/www.ncbi.nlm.nih.gov/BLAST). Sequence analysis of the PCR products identified the ampC gene as CMY-2.

### EXAMPLE 2

### Unusual Salmonella enterica serotype Typhimurium Isolate Producing CMY-7, SHV-9, and OXA-30 beta-lactamases

### Materials and Methods

Strain and Patient History. The S. enterica isolate was cultured from a stool sample of a 14 month old girl hospitalized for diarrhea and fever after a four week round trip from Australia to Pakistan, Turkey, and Saudi Arabia, in February and March, 2000. Stool culture yielded Campylobacter spp., Shigella boydii, Shigella flexneri, and the S. enterica isolate. The S. enterica isolate was serologically typed as S. enterica serotype typhimurium (4, 12:i:1,2), and was untypable by phage typing. It appeared to be unusual in its expression of resistance to cefoxitin, cefotaxime, ceftazidime, and aztreonam, with the aztreonam resistance being partially reversible by the addition of clavulanate.

Susceptibility testing and β-lactamase investigations. Antibiotic susceptibilities and resistance phenotypes were investigated by microdilution MIC methodology, double disk potentiation, VITEK (card GNS-424, bioMérieux, Inc. St. Louis, Mo.), NCCLS disk diffusion, and the three dimensional test (Thomson et al., Antimicrob. Agents Chemother., 35(5):1001-1003 (May 1991); and Thomson et al., Antimicrob. Agents Chemother., 36(9):1877-1882 (September 1992)).

Crude beta-lactamase preparations derived from sonicated bacterial cultures of the S. typhimurium isolate and strains producing reference beta-lactamases were assessed for beta-lactamase pls and general inhibitor characteristics by isoelectric focusing (Thomson et al., Antimicrob. Agents Chemother., 35(5):1001-1003 (May 1991).

Polymerase Chain Reaction (PCR). Template DNA was prepared by incubating for 20 hours at 37° C. with shaking. Cells from 1.3 milliliters of an overnight culture were harvested by centrifugation at 17,310×g in a microfuge for 5 minutes. The supernatant was decanted and the pellet was resuspended in 500microliters of distilled water. The cells were then lysed by heating at 95° C. for 10 minutes, and cellular debris was removed by centrifugation at 17,310×g in a microfuge for 5 minutes (Pitout et al., AAC 1998 42:1350-1354). PCR amplifications were carried out as previously described in Hanson et al., J. Antimicrob. Chemother., 44(3):377-380 (September 1999) using the following modifications: the final volume was 50 μL using 2 μL of template DNA with 0.5 mM primer. The annealing temperature and MgCl₂ preparation and PCR amplifications were carried out by inoculating the organisms into 5 milliliters of Luria-Bertani broth at concentrations for each primer set indicated with the appropriate primer set. The following oligonucleotide primer sets were used to PCR amplify the entire structural gene of the following beta-lactamase genes: bla_{OXA30}, prOXA30F and prOXA30R; bla_{CMY-7}, prCMY25F1 and prCMY2DR1; and bla_{SHV-9}, prSHV1F and prSHVDR (Table 1). The annealing temperature for primer sets amplifying bla_{CMY7} and bla_{OXA30} was 50° C. and for bla_{SHV}, 55° C. The MgCl₂ concentration was 2 mM for all PCR reactions. PCR products were generated at least two times and sequenced by automated PCR cycle-sequencing with dye-terminator chemistry using a DNA stretch sequencer from Applied Biosystems. The PCR products were directly sequenced except for the bla_{CMY-7} amplicon, which was gel-purified using a 1.5% agarose gel in tris-acetate EDTA buffer. DNA was extracted from the agarose using the Qiagen gel extraction kit (Qiagen, Valencia, Calif.). Additional primers used to obtain internal sequence are listed in Table 1 below

TABLE 1

| | | | | |
|---|---|---|---|---|
| Primer Name^{a} | Sequence^{b} | Corresponding (nts)^{c} | Function^{d} | Accession Number^{e} |

| | | | | | |
|---|---|---|---|---|---|
| CMY25F1 | CAATGTGTGAGAAGCAGTC | (SEQ ID NO:7) | 1736-1754 | Amp/Seq | X91840 |
| CMY2DR1 | CGCATGGGATTTTCCTTGCTG | (SEQ ID NO:8) | 3167-3147 | Amp/Seq | X91840 |
| OXA305F | GGAGCAGCAACGATGTTACG | (SEQ ID NO:1) | 1243-1262 Amp/Seq | AF255921 | |
| OXA303R | CGACTTGATTGAAGGGTTGG | (SEQ ID NO:2) | 2231-2212 | Amp/Seq | AF255921 |
| SHV1F | CACTCAAGGATGTATTGTG | (SEQ ID NO:33) | 99-117 | Amp/Seq | S82452 |
| SHVDR | CACCACCATCATTACCGACC | (SEQ ID NO:34) | 1081-1062 | Amp/Seq | S82452 |
| SHVEndR | TTAGCGTTGCCAGTGCTCG | (SEQ ID NO:35) | 978-960 | Seq | S82452 |
| CMY71F | CGTTATGCTGCGCTCTGCTG | (SEQ ID NO:9) | 1937-1956, | Seq | X91840/AJ011291 |
| | | | 14-33 | | |
| CMY71R | CTGCGGAACCGTAATCCAGG | (SEQ ID NO:10) | 2562-2543, | Seq | X91840/AJ011291 |
| | | | 639-620 | | |
| CFCF1 | CTGGCAACCACAATGGACTCCG | (SEQ ID NO:36) | 513-534 | Seq | X03866 |
| CFC1R | GCCAGTTCAGCATCTCCCAGCC | (SEQ ID NO:37) | 931-910 | Seq | X03866 |
| OXA1B14 | CGACCCCAAGTTTCCTGTAAGTG | (SEQ ID NO:38) | 2124-2102 | Seq | AF255921 |
| OXA1F2 | TGTGCAACGCAAATGGCAC | (SEQ ID NO:39) | 1546-1564 | Seq | AF255921 |

^{a}Primer name as it appears in the text.
^{b}Sequence of the primer used designated as 5′-3′.
^{c}Corresponding nucleotides in the referenced accession number which correspond to the 5′ to 3′ primer sequence.
^{d}Protocol for which primers were used: Amp, amplification; Seq, sequencing.
^{e}GenBank accession numbers from which primer sequence was derived

### Results

The isolate was multiply antibiotic resistant, being susceptible to only imipenem, meropenem, ciprofloxacin, and levofloxacin, and intermediate to cefepime (Table 2). The MIC of the investigational agent, faropenem, was 2 milligrams per liter (mg/l). It was resistant to other cephalosporins, penicillins, beta-lactam drug/blactamase inhibitor combinations, aminoglycosides, chloramphenicol, and cotrimoxazole. Phenotypic, inhibitor-based ESBL tests were positive, demonstrating potentiation of cefepime, cefpirome, and aztreonam by clavulanate. The 3-dimensional test indicated beta-lactamase-mediated hydrolysis of cefoxitin, a characteristic of AmpC beta-lactamases.

TABLE 2

| Agent | MIC |
|---|---|
| Ampicillin | >256 |
| Amoxicillin/clavulnate | 64 |
| Ticarcillin/clavulanate | >256 |
| Cephalothin | >32 |
| Cefixime | >128 |
| Cefprozil | >128 |
| Cefuroxime | >256 |
| Ceftriaxone | >128 |
| Ceftazidime | >32 |
| Faropenem | 2 |
| Imipenem | 0.25 |
| Meropenem | less than 2 |
| Ciprofloxacin | 0.125 |
| Levofloxacin | 0.5 |
| Amikacin | >64 |
| Gentamicin | >16 |
| Tobramycin | >16 |
| Chloramphenicol | >256 |
| Cotrimoxazole | >320 |

Isoelectric focusing revealed three beta-lactamases with pI values of 7.4, 8.2 and >9.0. These values were consistent with OXA-1 or OXA-30 (pI 7.4), SHV-9 (also known as SHV-5a) (pI 8.2), and AmpC (pI>9.0). The beta-lactamases with pI values 7.4 and 8.2 were inhibited by clavulanate but not cloxacillin, and the pI>9.0 beta-lactamase was inhibited by cloxacillin, but not clavulanate.

PCR amplification suggested the presence of a bla_{SHV}-like gene, a bla_{OXA}-like gene, and a bla_{CMY}-like ampC gene. Sequence data for each amplicon identified the genes as bla_{SHV-9} (Prinarakis et al., FEMS Microbiol. Lett., 139(2-3):229-234 (June 1996)), bla_{OXA-30} (Antimicrob. Agents Chemother., 44(8):2034-2038 (August 2000)) and bla_{CMY-7} (GenBank accession number AJ011291).

The complete disclosures of all patents, patent documents, publications, ATCC deposits, electronically available material (e.g., GenBank amino acid and nucleotide sequence submissions), etc., cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A primer selected from the group consisting of:
5′-GGAGCAGCAACGATGTTACG-3′ (SEQ ID NO:1);
5′-CGACTTGATTGAAGGGTTGG-3′ (SEQ ID NO:2); and full-length complements thereof.

2. A primer selected from the group consisting of:
5′-GCATTACTTCAGCTATGGGC-3′ (SEQ ID NO:3);
5′-GGCATTGGGATAGTTGCGGTTG-3′ (SEQ ID NO:4); and full-length complements thereof.

3. A primer selected from the group consisting of: 5′-CACCACGAGAATAACC-3′ (SEQ ID NO:5); 5′-GCCTTGAACTCGACCG-3′ (SEQ ID NO:6); and full-length complements thereof.

4. A primer selected from the group consisting of:
5′-CAATGTGTGAGAAGCAGTC-3′ (SEQ ID NO:7);
5′-CGCATGGGATTTTCCTTGCTG-3′ (SEQ ID NO:8);
5′-CGTTATGCTGCGCTCTGCTG-3′ (SEQ ID NO:9);
5′-CTGCGGAACCGTAATCCAGG-3′ (SEQ ID NO:10);
5′-CGTTATGCTGCGCTCTGC-3′ (SEQ ID NO:11);
5′-GGCGATATCGGCTTTACC-3′ (SEQ ID NO:12); and full-length complements thereof.

5. A primer selected from the group consisting of:
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16); and full-length complements thereof.

6. A primer selected from the group consisting of:
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20); and full-length complements thereof.

7. A primer selected from the group consisting of:
5′-CCTGCAACCTAAGAGAGCTTCT-3′ (SEQ ID NO:21);
5′-CGCGGTGGATGATGTGGTAA-3′ (SEQ ID NO:22); and full-length complements thereof.

8. A primer selected from the group consisting of:
5′-CCGGATGAGGTCAAGGATAACGC-3′ (SEQ ID NO:23);
5′-CCCCAGGCGTAATGCGCCTCTTCC-3′ (SEQ ID NO:24); and full-length complements thereof.

9. A primer selected from the group consisting of:
5′-GCTACACCTAGCTCCACCTTC-3′ (SEQ ID NO:25);
5′-GACAGTGGTTGGTAATCCATGC-3′ (SEQ ID NO:26);
5′-GTATCGCCGTCTAGTTCTGC-3′ (SEQ ID NO:27);
5′-GGTCGTGTTTCCCTTTAGCC-3′ (SEQ ID NO:28);
5′-GGTAATCTGGCACGCATGGT-3′ (SEQ ID NO:29);
5′-CACACTGAGCATATGCTGAC-3′ (SEQ ID NO:30);
5′-GGCCAATACAAAGGGCATCG-3′ (SEQ ID NO:31);
5′-CTACCCAATCGCTTGGTACG-3′ (SEQ ID NO:32); and full-length complements thereof.

10. A method for identifying a beta-lactamase nucleic acid in a clinical sample, the method comprising:
providing a clinical sample;
contacting the clinical sample with a pair of oligonucleotide primers specific for nucleic acid characteristic of the OXA-30 beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase nucleic acid.

11. The method of claim 10 wherein the primers are selected from the group consisting of:
5′-GGAGCAGCAACGATGTTACG-3′ (SEQ ID NO: 1);
5′-CGACTTGATTGAAGGGTTGG-3′ (SEQ ID NO:2); and full-length complements thereof.

12. A method for identifying a beta-lactamase nucleic acid in a clinical sample, the method comprising:
providing a clinical sample;
contacting the clinical sample with a pair of oligonucleotide primers having sequences 5′-GCATTACTTCAGCTATGGGC-3′ (SEQ ID NO:3), or a full-length complement thereof, and
5′-GGCATTGGGATAGTTGCGGTTG-3′ (SEQ ID NO:4), or a full-length complement thereof, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

13. A method for identifying a beta-lactamase nucleic acid in a clinical sample, the method comprising:
providing a clinical sample;
contacting the clinical sample with a pair of oligonucleotide primers having sequences 5′-CACCACGAGAATAACC-3′ (SEQ ID NO:5), or a full-length complement thereof, and 5′-GCCTTGAACTCGACCG-3′ (SEQ ID NO:6), or a full-length complement thereof, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

14. A method for identifying a beta-lactamase nucleic acid in a clinical sample, the method comprising:
providing a clinical sample;
contacting the clinical sample with a pair of oligonucleotide primers specific for nucleic acid characteristic of the FOX-5 beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase nucleic acid.

15. The method of claim 14 wherein the primers are selected from the group consisting of:
5′-CACCACGAGAATAACC-3′ (SEQ ID NO:5);
5′-GCCTTGAACTCGACCG-3′ (SEQ ID NO:6), and full-length complements thereof.

16. A method for identifying a beta-lactamase nucleic acid in a clinical sample, the method comprising:
providing a clinical sample;
contacting the clinical sample with a pair of oligonucleotide primers selected from the group consisting of 5′-CAATGTGTGAGAAGCAGTC-3′ (SEQ ID NO:7), 5′-CGCATGGGATTTTCCTTGCTG-3′ (SEQ ID NO:8), 5′-CGTTATGCTGCGCTCTGCTG-3′ (SEQ ID NO:9), 5′-CTGCGGAACCGTAATCCAGG-3′ (SEQ ID NO:10), 5′-CGTTATGCTGCGCTCTGC-3′ (SEQ ID NO:11), 5′- GGCGATATCGGCTTTACC-3′ (SEQ ID NO:12), and full-length complements thereof, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

17. A method for identifying a beta-lactamase nucleic acid in a clinical sample, the method comprising:
providing a clinical sample;
contacting the clinical sample with a pair of oligonucleotide primers specific for nucleic acid characteristic of the plasmid-mediated AmpC beta-lactamase enzymes derived from Morganella morganii, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

18. The method of claim 17 wherein the enzymes derived from Morganella morganii are selected from the group consisting of enzymes designated as DHA-1 and DHA-2.

19. The method of claim 17 wherein the primers are selected from the group consisting of:
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16); and full-length complements thereof.

20. A method for identifying a beta-lactamase nucleic acid in a clinical sample, the method comprising:
providing a clinical sample;
contacting the clinical sample with a pair of oligonucleotide primers specific for nucleic acid characteristic of the plasmid-mediated AmpC beta-lactamase enzymes derived from Hafnia alvei, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

21. The method of claim 20 wherein the enzymes derived from Hafnia alvei are enzymes designated as ACC-1.

22. The method of claim 20 wherein the primers are selected from the group consisting of:
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20); and full-length complements thereof.

23. A method for identifying a beta-lactamase nucleic acid in a clinical sample, the method comprising:
providing a clinical sample;
contacting the clinical sample with a pair of oligonucleotide primers having sequences 5′-CCTGCAACCTAAGAGAGCTTCT-3′ (SEQ ID NO:21), or a full-length complement thereof, and 5′-GCGCCTGGATGATGTGGTAA-3′ (SEQ ID NO:22), or a full-length complements thereof, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

24. A method for identifying a beta-lactamase nucleic acid in a clinical sample, the method comprising:
providing a clinical sample;
contacting the clinical sample with a pair of oligonucleotide primers specific for nucleic acid characteristic of the plasmid-mediated AmpC beta-lactamase enzymes designated as MIR-1 and ACT-1, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

25. The method of claim 24 wherein the primers are selected from the group consisting of:
5′-CCGGATGAGGTCAAGGATAACGC-3′ (SEQ ID NO:23);
5′-CCCCAGGCGTAATGCGCCTCTTCC-3′ (SEQ ID NO:24); and full-length complements thereof.

26. A method for identifying a beta-lactamase nucleic acid in a clinical sample, the method comprising:
providing a clinical sample;
contacting the clinical sample with a pair of oligonucleotide primers specific for nucleic acid characteristic of the plasmid-mediated AmpC beta-lactamase enzymes derived from Enterobacter cloacae, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase;
wherein the primers are selected from the group consisting of:
5′-CCGGATGAGGTCAAGGATAACGC-3′ (SEQ ID NO:23);
5′-CCCCAGGCGTAATGCGCCTCTTCC-3′ (SEQ ID NO:24); and full-length complements thereof.

27. A method for identifying a beta-lactamase nucleic acid in a clinical sample, the method comprising:
providing a clinical sample;
contacting the clinical sample with a pair of oligonucleotide primers specific for nucleic acid characteristic of an AmpC beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase;
wherein the primers are selected from the group consisting of:
5′-GCATTACTTCAGCTATGGGC-3′ (SEQ ID NO:3);
5′-GGCATTGGGATAGTTGCGGTTG-3′ (SEQ ID NO:4);
5′-CACCACGAGAATAACC-3′ (SEQ ID NO:5);
5′-GCCTTGAACTCGACCG-3′ (SEQ ID NO:6);
5′-CAATGTGTGAGAAGCAGTC-3′ (SEQ ID NO:7);
5′-CGCATGGGATTTTCCTTGCTG-3′ (SEQ ID NO:8);
5′-CGTTATGCTGCGCTCTGCTG-3′ (SEQ ID NO:9);
5′-CTGCGGAACCGTAATCCAGG-3′ (SEQ ID NO:10);
5′-CGTTATGCTGCGCTCTGC-3′ (SEQ ID NO:11);
5′-GGCGATATCGGCTTTACC-3′ (SEQ ID NO:12);
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16);
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20);
5′-CCTGCAACCTAAGAGAGCTTCT-3′ (SEQ ID NO:21);
5′-GCGCCTGGATGATGTGGTAA-3′ (SEQ ID NO:22);
5′-CCGGATGAGGTCAAGGATAACGC-3′ (SEQ ID NO:23);
5′-CCCCAGGCGTAATGCGCCTCTTCC-3′ (SEQ ID NO:24); and full-length complements thereof.

28. A method for identifying a beta-lactamase nucleic acid in a clinical sample, the method comprising:
providing a clinical sample;
contacting the clinical sample with a pair of oligonucleotide primers specific for nucleic acid characteristic of a beta-lactamase enzyme, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase;
wherein the primers are selected from the group consisting of:
5′-GGAGCAGCAACGATGTTACG-3′ (SEQ ID NO:1);
5′-CGACTTGATTGAAGGGTTGG-3′ (SEQ ID NO:2);
5′-GCATTACTTCAGCTATGGGC-3′ (SEQ ID NO:3);
5′-GGCATTGGGATAGTTGCGGTTG-3′ (SEQ ID NO:4);
5′-CACCACGAGAATAACC-3′ (SEQ ID NO:5);
5′-GCCTTGAACTCGACCG-3′ (SEQ ID NO:6);
5′-CAATGTGTGAGAAGCAGTC-3′ (SEQ ID NO:7);
5′-CGCATGGGATTTTCCTTGCTG-3′ (SEQ ID NO:8);
5′-CGTTATGCTGCGCTCTGCTG-3′ (SEQ ID NO:9);
5′-CTGCGGAACCGTAATCCAGG-3′ (SEQ ID NO:10);
5′-CGTTATGCTGCGCTCTGC-3′ (SEQ ID NO:11);
5′-GGCGATATCGGCTTTACC-3′ (SEQ ID NO:12);
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16);
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20);
5′-CCTGCAACCTAAGAGAGCTTCT-3′ (SEQ IDNO:21);
5′-GCGCCTGGATGATGTGGTAA-3′ (SEQ ID NO:22);
5′-CCGGATGAGGTCAAGGATAACGC-3′ (SEQ ID NO:23);
5′-CCCCAGGCGTAATGCGCCTCTTCC-3′ (SEQ ID NO:24);
5′-GCTACACCTAGCTCCACCTTC-3′ (SEQ ID NO:25);
5′-GACAGTGGTTGGTAATCCATGC-3′ (SEQ ID NO:26);
5′-GTATCGCCGTCTAGTTCTGC-3′ (SEQ ID NO:27);
5′-GGTCGTGTTTCCCTTTAGCC-3′ (SEQ ID NO:28);
5′-GGTAATCTGGCACGCATGGT-3′ (SEQ ID NO:29);
5′-CACACTGAGCATATGCTGAC-3′ (SEQ ID NO:30);
5′-GGCCAATACAAAGGGCATCG-3′ (SEQ ID NO:31);
5′-CTACCCAATCGCTTGGTACG-3′ (SEQ ID NO:32); and full-length complements thereof.

29. A method for identifying a beta-lactamase nucleic acid in a clinical sample, the method comprising:
providing a clinical sample;
contacting the clinical sample with a pair of oligonucleotide primers specific for nucleic acid characteristic of carbapenem-hydrolyzing beta-lactamase enzymes, wherein one primer of the pair is complementary to at least a portion of the beta-lactamase nucleic acid in the sense strand and the other primer of each pair is complementary to at least a portion of the beta-lactamase nucleic acid in the antisense strand;
annealing the primers to the beta-lactamase nucleic acid;
simultaneously extending the annealed primers from a 3′ terminus of each primer to synthesize an extension product that is complementary to the nucleic acid strands annealed to each primer wherein each extension product after separation from the beta-lactamase nucleic acid serves as a template for the synthesis of an extension product for the other primer of each pair;
separating the amplified products; and
analyzing the separated amplified products for a size characteristic of the beta-lactamase.

30. The method of claim 29 wherein at least one of the primers of the primer pair is selected from the group consisting of:
5′-GCTACACCTAGCTCCACCTTC-3′ (SEQ ID NO:25);
5′-GACAGTGGTTGGTAATCCATGC-3′ (SEQ ID NO:26);
5′-GTATCGCCGTCTAGTTCTGC-3′ (SEQ ID NO:27);
5′-GGTCGTGTTTCCCTTTAGCC-3′ (SEQ ID NO:28);
5′-GGTAATCTGGCACGCATGGT-3′ (SEQ ID NO:29);
5′-CACACTGAGCATATGCTGAC-3′ (SEQ ID NO:30);
5′-GGCCAATACAAAGGGCATCG-3′ (SEQ ID NO:31);
5′-CTACCCAATCGCTTGGTACG-3′ (SEQ ID NO:32); and full-length complements thereof.

31. A diagnostic kit for detecting an OXA-30 beta-lactamase nucleic acid, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein the primers are selected from the group consisting of:
5′- GGAGCAGCAACGATGTTACG - 3′ (SEQ ID NO:1);
5′-CGACTTGATTGAAGGGTTGG -3′ (SEQ ID NO:2); and full-length complements thereof.

32. A diagnostic kit for detecting an AmpC family beta-lactamase, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein at least one of the primers of the primer pair is selected from the group consisting of:
5′-GCATTACTTCAGCTATGGGC-3′ (SEQ ID NO:3);
5′-GGCATTGGGATAGTTGCGGTTG-3′ (SEQ ID NO:4);
5′-CACCACGAGAATAACC-3′ (SEQ ID NO:5);
5′-GCCTTGAACTCGACCG-3′ (SEQ ID NO:6);
5′-CAATGTGTGAGAAGCAGTC-3′ (SEQ ID NO:7);
5′-CGCATGGGATTTTCCTTGCTG-3′ (SEQ ID NO:8);
5′-CGTTATGCTGCGCTCTGCTG-3′ (SEQ ID NO:9);
5′-CTGCGGAACCGTAATCCAGG-3′ (SEQ ID NO:10);
5′-CGTTATGCTGCGCTCTGC-3′ (SEQ ID NO:11);
5′-GGCGATATCGGCTTTACC-3′ (SEQ ID NO:12);
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16);
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20);
5′-CCTGCAACCTAAGAGAGCTTCT-3′ (SEQ ID NO:21);
5′-GCGCCTGGATGATGTGGTAA-3′ (SEQ ID NO:22);
5′-CCGGATGAGGTCAAGGATAACGC-3′ (SEQ ID NO:23);
5′-CCCCAGGCGTAATGCGCCTCTTCC-3′ (SEQ ID NO:24); and full-length complements thereof.

33. A diagnostic kit for detecting a carbapenem-hydrolyzing beta-lactamase, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid.

34. The diagnostic kit of claim 33 wherein the primers are selected from the group consisting of:
5′-GCTACACCTAGCTCCACCTTC-3′ (SEQ ID NO:25);
5′-GACAGTGGTTGGTAATCCATGC-3′ (SEQ ID NO:26);
5′-GTATCGCCGTCTAGTTCTGC-3′ (SEQ ID NO:27);
5′-GGTCGTGTTTCCCTTTAGCC-3′ (SEQ ID NO:28);
5′-GGTAATCTGGCACGCATGGT-3′ (SEQ ID NO:29);
5′-CACACTGAGCATATGCTGAC-3′ (SEQ ID NO:30);
5′-GGCCAATACAAAGGGCATCG-3′ (SEQ ID NO:31);
5′-CTACCCAATCGCTTGGTACG-3′ (SEQ ID NO:32); and full-length complements thereof.

35. A diagnostic kit for detecting a beta-lactamase nucleic acid, wherein the kit comprises:
(a) at least one primer pair capable of hybridizing to a beta-lactamase nucleic acid;
(b) at least one positive control and at least one negative control; and
(c) a protocol for identification of the beta-lactamase nucleic acid, wherein the primers are selected from the group consisting of:
5′-GGAGCAGCAACGATGTTACG-3′ (SEQ ID NO:1);
5′-CGACTTGATTGAAGGGTTGG-3′ (SEQ ID NO:2);
5′-GCATTACTTCAGCTATGGGC-3′ (SEQ ID NO:3);
5′-GGCATTGGGATAGTTGCGGTTG-3′ (SEQ ID NO:4);
5′-CACCACGAGAATAACC-3′ (SEQ ID NO:5);
5′-GCCTTGAACTCGACCG-3′ (SEQ ID NO:6);
5′-CAATGTGTGAGAAGCAGTC-3′ (SEQ ID NO:7);
5′-CGCATGGGATTTTCCTTGCTG-3′ (SEQ ID NO:8);
5′-CGTTATGCTGCGCTCTGCTG-3′ (SEQ ID NO:9);
5′-CTGCGGAACCGTAATCCAGG-3′ (SEQ ID NO:10);
5′-CGTTATGCTGCGCTCTGC-3′ (SEQ ID NO:11);
5′-GGCGATATCGGCTTTACC-3′ (SEQ ID NO:12);
5′-CCGTTACTCACACACGGAAGG-3′ (SEQ ID NO:13);
5′-CGTATCCGCAGGGGCCTGTTC-3′ (SEQ ID NO:14);
5′-GCGTCTGTATGCAAACAGCAG-3′ (SEQ ID NO:15);
5′-CAATGCGACCTCGTTGGTCACG-3′ (SEQ ID NO:16);
5′-CCGATTAAAAGGTCAC-3′ (SEQ ID NO:17);
5′-GTGACTCAACATATCG-3′ (SEQ ID NO:18);
5′-CGTTAGCGTACTCAATGTGG-3′ (SEQ ID NO:19);
5′-GATCCTGAGTAATCTCACCC-3′ (SEQ ID NO:20);
5′-CCTGCAACCTAAGAGAGCTTCT-3′ (SEQ IDNO:21);
5′-GCGCCTGGATGATGTGGTAA-3′ (SEQ ID NO:22);
5′-CCGGATGAGGTCAAGGATAACGC-3′ (SEQ ID NO:23);
5′-CCCCAGGCGTAATGCGCCTCTTCC-3′ (SEQ ID NO:24);
5′-GCTACACCTAGCTCCACCTTC-3′ (SEQ ID NO:25);
5′-GACAGTGGTTGGTAATCCATGC-3′ (SEQ ID NO:26);
5′-GTATCGCCGTCTAGTTCTGC-3′ (SEQ ID NO:27);
5′-GGTCGTGTTTCCCTTTAGCC-3′ (SEQ ID NO:28);
5′-GGTAATCTGGCACGCATGGT-3′ (SEQ ID NO:29);
5′-CACACTGAGCATATGCTGAC-3′ (SEQ ID NO:30);
5′-GGCCAATACAAAGGGCATCG-3′ (SEQ ID NO:31);
5′-CTACCCAATCGCTTGGTACG-3′ (SEQ ID NO:32); and full-length complements thereof.


SEQUENCE LISTING (39 sequences)

SEQ ID NO: 1 - DNA, 20 bp, ARTIFICIAL
  Primer
ggagcagcaacgatgttacg

SEQ ID NO: 2 - DNA, 20 bp, ARTIFICIAL
  Primer
cgacttgattgaagggttgg

SEQ ID NO: 3 - DNA, 20 bp, ARTIFICIAL
  Primer
gcattacttcagctatgggc

SEQ ID NO: 4 - DNA, 22 bp, ARTIFICIAL
  Primer
ggcattgggatagttgcggttg

SEQ ID NO: 5 - DNA, 16 bp, ARTIFICIAL
  Primer
caccacgagaataacc

SEQ ID NO: 6 - DNA, 16 bp, ARTIFICIAL
  Primer
gccttgaactcgaccg

SEQ ID NO: 7 - DNA, 19 bp, ARTIFICIAL
  Primer
caatgtgtgagaagcagtc

SEQ ID NO: 8 - DNA, 21 bp, ARTIFICIAL
  Primer
cgcatgggattttccttgctg

SEQ ID NO: 9 - DNA, 20 bp, ARTIFICIAL
  Primer
cgttatgctgcgctctgctg

SEQ ID NO: 10 - DNA, 20 bp, ARTIFICIAL
  Primer
ctgcggaaccgtaatccagg

SEQ ID NO: 11 - DNA, 18 bp, ARTIFICIAL
  Primer
cgttatgctgcgctctgc

SEQ ID NO: 12 - DNA, 18 bp, ARTIFICIAL
  Primer
ggcgatatcggctttacc

SEQ ID NO: 13 - DNA, 21 bp, ARTIFICIAL
  Primer
ccgttactcacacacggaagg

SEQ ID NO: 14 - DNA, 21 bp, ARTIFICIAL
  Primer
cgtatccgcaggggcctgttc

SEQ ID NO: 15 - DNA, 21 bp, ARTIFICIAL
  Primer
gcgtctgtatgcaaacagcag

SEQ ID NO: 16 - DNA, 22 bp, ARTIFICIAL
  Primer
caatgcgacctcgttggtcacg

SEQ ID NO: 17 - DNA, 16 bp, ARTIFICIAL
  Primer
ccgattaaaaggtcac

SEQ ID NO: 18 - DNA, 16 bp, ARTIFICIAL
  Primer
gtgactcaacatatcg

SEQ ID NO: 19 - DNA, 20 bp, ARTIFICIAL
  Primer
cgttagcgtactcaatgtgg

SEQ ID NO: 20 - DNA, 20 bp, ARTIFICIAL
  Primer
gatcctgagtaatctcaccc

SEQ ID NO: 21 - DNA, 22 bp, ARTIFICIAL
  Primer
cctgcaacctaagagagcttct

SEQ ID NO: 22 - DNA, 20 bp, ARTIFICIAL
  Primer
cgcggtggatgatgtggtaa

SEQ ID NO: 23 - DNA, 23 bp, ARTIFICIAL
  Primer
ccggatgaggtcaaggataacgc

SEQ ID NO: 24 - DNA, 24 bp, ARTIFICIAL
  Primer
ccccaggcgtaatgcgcctcttcc

SEQ ID NO: 25 - DNA, 21 bp, ARTIFICIAL
  Primer
gctacacctagctccaccttc

SEQ ID NO: 26 - DNA, 22 bp, ARTIFICIAL
  Primer
gacagtggttggtaatccatgc

SEQ ID NO: 27 - DNA, 20 bp, ARTIFICIAL
  Primer
gtatcgccgtctagttctgc

SEQ ID NO: 28 - DNA, 20 bp, ARTIFICIAL
  Primer
ggtcgtgtttccctttagcc

SEQ ID NO: 29 - DNA, 20 bp, ARTIFICIAL
  Primer
ggtaatctggcacgcatggt

SEQ ID NO: 30 - DNA, 20 bp, ARTIFICIAL
  Primer
cacactgagcatatgctgac

SEQ ID NO: 31 - DNA, 20 bp, ARTIFICIAL
  Primer
ggccaatacaaagggcatcg

SEQ ID NO: 32 - DNA, 20 bp, ARTIFICIAL
  Primer
ctacccaatcgcttggtacg

SEQ ID NO: 33 - DNA, 19 bp, ARTIFICIAL
  Primer
cactcaaggatgtattgtg

SEQ ID NO: 34 - DNA, 20 bp, ARTIFICIAL
  Primer
caccaccatcattaccgacc

SEQ ID NO: 35 - DNA, 19 bp, ARTIFICIAL
  Primer
ttagcgttgccagtgctcg

SEQ ID NO: 36 - DNA, 22 bp, ARTIFICIAL
  Primer
ctggcaaccacaatggactccg

SEQ ID NO: 37 - DNA, 22 bp, ARTIFICIAL
  Primer
gccagttcagcatctcccagcc

SEQ ID NO: 38 - DNA, 23 bp, ARTIFICIAL
  Primer
cgaccccaagtttcctgtaagtg

SEQ ID NO: 39 - DNA, 19 bp, ARTIFICIAL
  Primer
tgtgcaacgcaaatggcac